(19) United States
(12) Patent Application Publication
(10) Pub. No.: US 2023/0151445 A1
(43) Pub. Date: May 18, 2023

(54) METHOD FOR THE DETECTION AND QUANTIFICATION OF HUMAN CYTOMEGALOVIRUS BY MEANS OF VIRION RNAS

(71) Applicant: ELITECHGROUP S.P.A., MILANO (IT)

(72) Inventors: Eleonora MASTRAPASQUA, MILANO (IT); Guglielmo STEFANUTO, TORINO (IT); Gabriella CAMPANALE, NOLE (IT)

(21) Appl. No.: 17/995,642

(22) PCT Filed: Mar. 31, 2021
(86) PCT No.: PCT/EP2021/058497

§ 371 Date: Oct. 6, 2022

(30) Foreign Application Priority Data
Apr. 7, 2020 (IT) 102020000007357

(51) Int. Cl. C12Q 1/70; C12Q 1/686

(52) U.S. Cl. CPC C12Q 1/705; C12Q 1/686

(57) ABSTRACT

A method for detection and/or quantification of a human cytomegalovirus (hCMV) virion mRNA in a sample obtained from a patient to identify and/or quantify hCMV infection includes: i) optionally removing from the sample any cellular component that might be present, obtaining a cell-free sample; ii) extracting RNA from a cell-free sample obtained from the patient or obtained in step i); iii) forming a reaction mixture by contacting said RNA extracted in step ii); iv) subjecting the reaction mixture to a reverse transcription process under conditions suitable to generate a cDNA corresponding to the hCMV virion mRNA; v) subjecting the cDNA to an amplification process under conditions adapted to generate an amplification product constituted by DNA; and vi) detecting a presence and/or quantifying the quantity of the amplification product constituted by DNA, as an indication of the presence and/or of the quantity of hCMV virion mRNA in the sample.

### TECHNICAL FIELD

The present disclosure relates to a method for the detection and/or quantification of human cytomegalovirus (hCMV) in a sample obtained from a patient, and to primers, probes and kits useful in said method.

### SEQUENCE LISTING

The Instant Application contains a Sequence Listing which has been submitted electronically in ASCII format and is hereby incorporated by reference in its entirety. Said ASCII copy, created on Oct. 5, 2022 is named “MOD1588US_ST25” and is 38,775 bytes in size.

### BACKGROUND

Despite progress in clinical practice and treatment strategies, hCMV infection is still a significant problem especially in the field of transplants, since it is capable of strongly affecting the result of the transplant, both in hematopoietic stem cell transplantation (HSCT) (Azevedo et al., Clinics 2015; 70(7):515-523) and in solid organ transplantation (SOT) (Andrews et al., Transplantation 2011; 92(11):1181-7). This is mainly due to the combination of a high prevalence of hCMV in the population (Bate et al., Clin Infect Dis. 2010; 50(11):1439-47; Beam et al., Curr Infect Dis Rep. 2012; 14(6):633-41) and of post-transplantation immunosuppression therapies which expose patients to a higher risk of developing severe hCMV-related diseases, both at the organ level and at the systemic level, and can also be associated with transplant rejection (Torre-Cisneros et al., Clin Infect Dis. 2009; 49(8):1167-8; Crough et al., Clin Microbiol Rev. 2009; 22(1):76-98).

The necessary management of hCMV infection is currently performed essentially with two different but correlated strategies: preemptive therapy and universal prophylaxis (Andrews et al., Transplantation 2011; 92(11):1181-7; Emery et al., 2013 Br J Haematol. 2013; 162(1):25-39; Kotton et al., Transplantation 2013; 96(4):333-60). Universal prophylaxis consists in administering to all the patients an antiviral agent (such as for example ganciclovir or valganciclovir) for a certain period of time after transplantation. Preemptive therapy consists in treating only patients with infection and only above a certain viral load defined initially by means of viral culture methods (viremia), then by means of white blood cell immunofluorescence methods (antigenemia) and currently by means of molecular biology methods (nucleic acid amplification technologies). Both approaches are based on the administration of drugs which inhibit the DNA replication mechanism. During treatments, monitoring of the viral load by quantification of hCMV DNA (DNAemia) in whole blood or in plasma by means of the realtime polymerase chain reaction has been recognized as the best clinical practice, and as such it is recommended and approved in various current international guidelines (Kotton et al., Transplantation 2018; 102(6):900-931, Griffiths et al., PLoS One. 2016; 11(9):e0163722, Emery et al., 2013 Br J Haematol. 2013; 162(1):25-39). Monitoring of DNAemia by means of realtime PCR is based on the application of reference thresholds of the quantity of hCMV DNA for a clinical procedure and on the description of the hCMV replication kinetics. Quantitative realtime PCR methods make it possible to obtain an accurate and precise quantification of the viral DNA in a broad measurement interval.

Current antiviral drugs, including ganciclovir, aciclovir, valganciclovir, valaciclovir, foscarnet and cidofovir, have demonstrated their high effectiveness in the treatment of hCMV infections (McIntosh et al., J Virus Erad. 2016; 2(3):143-8, Emery et al., 2013 Br J Haematol. 2013; 162(1):25-39, Ljungman et al., Hematol Oncol Clin North Am. 2011; 25(1):151-69), but they entail a high toxicity. Moreover, prolonged exposure to low dosages can induce the development of drug-resistant hCMV strains. Accordingly, more effective and less toxic drugs have been studied in order to limit the exposure of the patients to the known adverse pharmacological effects. Among these, inhibitors of the terminase complex have been studied, among which letermovir has proved to be particularly effective (Lischka et al., Antimicrob Agents Chemother. 2010; 54(3):1290-7; Goldner et al., J. Virol. 2011; 85(20):10884-93; Chemaly et al., N Engl J Med. 2014; 370(19):1781-89, Mendelez et al., Infect Drug Resist. 2015; 8:269-77) and has received approval for patients subjected to hematopoietic stem cell transplantation (HSCT) (Marty et al., N Engl J Med. 2017; 377(25):2433-2444). The preliminary results of phase II studies also encourage their use in solid organ transplant (SOT) recipients (Stoelben et al., Transpl Int. 2014; 27(1):77-864). Letermovir is an antiviral agent that inhibits hCMV DNA maturation in single genomes but does not interfere with viral DNA replication. Letermovir in fact binds to the terminase complex components (UL51, UL56 and UL89) and blocks the cleavage of the DNA concatemer, causing the accumulation of hCMV DNA in the infected cells, which then die by apoptosis. One consequence of the accumulation of hCMV DNA in patients treated with letermovir is that current viral load monitoring methods based on the detection and quantification of hCMV DNA and on comparison with the reference thresholds are no longer adequate for monitoring viremia in post-transplantation follow-up (Bowman et al., Expert Opin Investig Drugs. 2017; 26(2):235-241). In this situation, one should revert to monitoring the hCMV load with viremia, the culture-based method for identifying the infectious viral particles that are present in the sample of the patient. However, this diagnostic method has been abandoned due to its complexity in execution and interpretation with respect to realtime PCR.

An alternative target to hCMV DNA for monitoring patients and in particular those immunodepressed or immunosuppressed treated with the aforementioned new drugs such as letermovir might be the messenger RNA (mRNA) of hCMV. Indeed, from the earliest stages of hCMV infection, messenger RNAs transcribed from the viral genome appear in infected cells. Moreover, it has been demonstrated that various viral mRNAs are incorporated in the virion (Bresnahan et al., Science 2000; 288(5475): 2373-6, Greijer et al., J. Virol. 2000; 74(19): 9078-82, Terhune et al., J Virol. 2004; 78(19): 10390-8).

The mRNA of hCMV contained in infected cells has been previously correlated directly with the onset of active infection starting from the analysis of samples of peripheral or medullary whole blood containing white cells infected by the virus. Among viral transcripts, immediate early mRNAs (IE mRNAs), coded for example by the UL123 gene, are relevant for the initial phase of the infection. Ultimately, the increase in IE mRNA is correlated with the progression of the infection and requires careful clinical assessment (Greijer et al., J Clin Virol. 2002; 24(1-2):57-66). The presence of late mRNAs such as the one for the pp67 protein instead indicates the final stage of a productive infection which requires immediate antiviral treatment. This monitoring of the level of mRNA was performed using an amplification technology known as “Nucleic Acid Sequence Based Amplification” (NASBA) in whole blood samples and demonstrated a sensitivity of approximately 104 copies/mL of RNA and a reproducible linearity in a measurement interval comprised between 104 and 107 copies/mL (Greijer et al., J Clin Virol. 2002; 24(1-2):57-66).

Various hCMV RNAs have been studied as targets in HSCT and SOT patients treated with ganciclovir and foscarnet. Again by using NASBA on whole blood samples, only IE mRNA showed a specificity and kinetics comparable with the DNAemia of hCMV (Gerna et al., Int J Antimicrob Agents. 2000; 16(4):455-60). Moreover, IE mRNA, if compared to other targets, correlates more reliably with an active infection in association with antiviral treatments (Gerna et al., Blood 2003; 101(12):5053-60). The DNA can in fact be detected even in the absence of active infection, while proteins such as pp65 can accumulate even with current treatments based on hCMV polymerase inhibitors (e.g., ganciclovir, foscarnet, valganciclovir) which do not interfere with protein synthesis.

However, the methods presented so far that use viral mRNA as a target have been applied to whole blood samples (i.e., cellular samples) in order to detect viral mRNA contained in white cells. Therefore, these methods are not aimed at monitoring circulating hCMV, which is the most specific indicator of active hCMV infection.

### SUMMARY

Therefore there remains a need for new quantitative methods for the detection of hCMV in patients treated with antivirals that cause accumulation of viral DNA (such as for example viral terminase complex inhibitors like letermovir) which have a sensitivity comparable with that obtained with current methods that use hCMV genomic DNA as target, but at the same time are not influenced by said hCMV genomic DNA.

In view of the limitations of the background art described above, the aim of the present disclosure is to provide a method for the detection and/or quantification of human cytomegalovirus (hCMV) as circulating viral particles (virions) in a sample obtained from a patient, in particular if he/she is immunodepressed or immunosuppressed, being treated with an antiviral drug capable of interfering with the cleavage and encapsidation of hCMV genomic DNA.

Within the scope of this aim, the disclosure provides a method for the detection and/or the quantification of human cytomegalovirus (hCMV) based on viral RNA incorporated in the circulating viral particle which has a sensitivity comparable to the one obtained with methods based on viral genomic DNA in immunodepressed or immunosuppressed patients treated with the antivirals used so far and a higher specificity, since it is able to be unaffected by interference by circulating free-form hCMV genomic DNA (produced by lysis of the cells infected by the virus).

The present disclosure provides oligonucleotides that are specific for virion mRNAs associated with active hCMV infection.

the present disclosure also provides a kit for the detection and/or quantification of hCMV that is capable of providing the clinician with diagnostic information on the presence of circulating virions with all the advantages of molecular biology methods (nucleic acid amplification technologies).

This aim, as well as these and other advantages which will become more apparent hereinafter, are achieved by providing a method for the detection and/or quantification of a virion mRNA of human cytomegalovirus (hCMV) in a sample obtained from a patient treated with an antiviral drug capable of interfering with the cleavage and encapsidation of hCMV DNA, said method comprising the steps of:

i) optionally removing from the sample obtained from the patient any cellular component that might be present, obtaining a cell-free sample;

ii) extracting RNA from a cell-free sample obtained from the patient or from the cell-free sample obtained in step i);

iii) forming a reaction mixture by contacting said RNA extracted in step ii) with a solution comprising:

d) one or more primer pairs specific for said hCMV virion mRNA;

e) an RNA-dependent DNA polymerase;

f) a DNA polymerase;

iv) subjecting said reaction mixture to a reverse transcription process under conditions suitable to generate a cDNA corresponding to said hCMV virion mRNA;

v) subjecting said cDNA to a DNA amplification process under conditions suitable to generate at least one amplification product constituted by DNA;

vi) detecting the presence and/or quantifying the quantity of said at least one amplification product constituted by DNA, as an indication of the presence and/or of the quantity of hCMV virion mRNA in the sample obtained from the patient.

The aim and advantages of the present disclosure are also achieved by providing an oligonucleotide consisting of a sequence selected from the group consisting of SEQ ID NOs: 1-116.

Finally, the aim and advantages of the present disclosure are also achieved by providing a kit for the detection and/or quantification of hCMV virion mRNA comprising:

a) at least one primer pair selected from:
a primer pair from a first set (“set 1)”) wherein a first primer has a sequence selected from the group consisting of SEQ ID NOs: 1-12 and a second primer has a sequence selected from the group consisting of SEQ ID NOs: 13-45; or
a primer pair from a second set (“set 2)”) wherein a first primer has a sequence selected from the group consisting of SEQ ID NOs: 50-71 and a second primer has a sequence selected from the group consisting of SEQ ID NOs: 72-84; or
a primer pair from a third set (“set 3)”) wherein a first primer has a sequence selected from the group consisting of SEQ ID NOs: 92-105 and a second primer has a sequence selected from the group consisting of SEQ ID NOs: 106-111; and

optionally

b) an RNA-dependent DNA polymerase; and

c) a DNA polymerase.

Further characteristics and advantages of the disclosure will become apparent from the detailed description that follows.

### DETAILED DESCRIPTION

The following definitions are applied in the description and in the claims.

An “amplification process” refers to any chemical reaction, including enzyme-based ones, that gives rise to an increase in copies of a DNA sequence. One method for DNA amplification is polymerase chain reaction (PCR), which is well-known to the person skilled in the art and is described for example in patent no. U.S. Pat. No. 4,683,202. Further amplification reactions comprise, among others, ligase chain reaction (LCR), polymerase and ligase chain reaction (PLCR), Gap-LCR, Strand Displacement Amplification (SDA), Rolling Circle Amplification (RCA) and loop-mediated isothermal amplification (LAMP).

The term “oligonucleotide” refers to molecules comprising two or more deoxyribonucleotides or ribonucleotides, such as for example primers, probes, fragments of nucleic acids to be detected and control nucleic acids. Oligonucleotides can be prepared by means of any suitable method known in the art, including for example cloning and restriction of appropriate sequences and direct chemical synthesis, such as the conventional and well-known chemistry of phosphoramidites. In the context of the present disclosure, the oligonucleotides can be chemically modified, i.e., the primer and/or the probe can comprise one or more modified nucleotides or non-nucleotide compounds.

The term “primer” is used here as known to the person skilled in the art and refers to natural or synthetic oligonucleotides capable of acting as a starting point for DNA synthesis in the conditions in which the synthesis of a primer extension product that is complementary to a nucleic acid filament is induced, i.e., in the presence of four different triphosphate nucleosides and of a polymerization agent (such as for example a DNA polymerase or reverse transcriptase enzyme) in an appropriate buffer and at a suitable temperature.

The term “probe” refers to natural or synthetic oligonucleotides capable of hybridizing, under the appropriate conditions, to an amplification product of a nucleic acid in order to detect that amplification product.

The term “sample” refers to a material obtained from a human patient, in particular a human patient treated with an antiviral drug capable of interfering with the cleavage and encapsidation of hCMV DNA, which is suspected of containing or potentially containing at least one human cytomegalovirus (hCMV) virion mRNA.

The term “cellular component” refers to the corpusculate part, constituted by cells or cell fragments, that can be present in a sample obtained from a patient.

In a first aspect, the present disclosure relates to a method for the detection and/or quantification of a human cytomegalovirus (hCMV) virion mRNA in a sample obtained from a patient treated with an antiviral drug capable of interfering with the cleavage and encapsidation of hCMV DNA, said method comprising the steps of:

i) optionally removing any cellular component that might be present from the sample obtained from the patient, obtaining a cell-free sample;

ii) extracting RNA from a cell-free sample obtained from the patient or from the cell-free sample obtained in step i);

iii) forming a reaction mixture by contacting said RNA extracted in step ii) with a solution comprising:

a) one or more primer pairs specific for said hCMV virion mRNA;

b) an RNA-dependent DNA polymerase;

c) a DNA polymerase;

iv) subjecting said reaction mixture to a reverse transcription process under conditions suitable to generate a cDNA corresponding to said hCMV virion mRNA;

v) subjecting said cDNA to an amplification process under conditions such as to generate an amplification product constituted by DNA;

vi) detecting the presence and/or quantifying the quantity of said at least one amplification product constituted by DNA, as an indication of the presence and/or of the quantity of hCMV virion mRNA in the sample obtained from the patient.

In one embodiment of the method according to the disclosure, the patient is immunodepressed or immunosuppressed.

If the sample obtained from patient is not cell-free at the source, the method according to the disclosure provides for a first step aimed at obtaining a cell-free sample, in order to be able to focus the analysis only on the virion mRNA transcribed by the hCMV genome and carried by the virions. The sample can therefore be a cell-free clinical sample or, for example in the case of whole blood, undergo a treatment (step (i) of the method according to the disclosure) to remove the cellular component. The methods for removing from clinical samples the cells that constitute the cellular component are known to the person skilled in the art and comprise, for example, whole blood centrifugation to obtain plasma, whole blood coagulation to obtain serum, centrifugation of bronchoalveolar lavages to obtain a supernatant, and others.

Preferably, the sample obtained from the patient is selected from the group consisting of plasma (optionally containing an anticoagulant), serum, urine, saliva, oral swab supernatant, cerebrospinal fluid (CSF), bronchoalveolar lavages (BAL), nasopharyngeal lavages, nasopharyngeal aspirates, pharyngeal swab supernatant, tear fluid, vitreous humor, eye swab supernatant, and fecal supernatant.
Preferably, the hCMV virion mRNA is an mRNA of a gene or of a coding region (ORF or CDS) of hCMV selected from the group consisting of UL21.5 (SEQ ID NO: 117), UL65 (SEQ ID NO: 122), UL83 (SEQ ID NO: 123), UL106 (SEQ ID NO: 124), UL107 (SEQ ID NO: 125), UL108 (SEQ ID NO: 126), UL109 (SEQ ID NO: 127), UL110 (SEQ ID NO: 128), UL122 (SEQ ID NO: 129), UL123 (SEQ ID NO: 130), TRL/IRL2 (SEQ ID NO: 131), TRL/IRL3 (SEQ ID NO: 132), TRL/IRL4 (SEQ ID NO: 133), TRL/IRL5 (SEQ ID NO: 134), TRL/IRL7 (SEQ ID NO: 135), and TRL/IRL13 (SEQ ID NO: 136).

To avoid or at least limit reactivity with DNA present in the sample, the primers specific for hCMV virion mRNA are preferably designed with their 5′ region complementary to a mRNA exon and the 3′ region complementary to an adjacent exon. In this manner it is possible to obtain primers which, under the reaction conditions, can hybridize only to mature mRNAs, i.e., mRNA from which, after transcription, the introns were removed and the exons have been spliced, or to the corresponding cDNAs. As a non-limiting example, the inventors of the present disclosure have designed primers specific for the mRNA of the UL21.5 gene of hCMV (astride the junction region of exons 1 and 2).

In a preferred embodiment of the method according to the disclosure, each of said one or more primer pairs is selected independently from:

a primer pair from a first set (“set 1)”) wherein a first primer has a sequence selected from the group consisting of SEQ ID NOs: 1-12 and a second primer has a sequence selected from the group consisting of SEQ ID NOs: 13-45; or

a primer pair from a second set (“set 2)”) wherein a first primer has a sequence selected from the group consisting of SEQ ID NOs: 50-71 and a second primer has a sequence selected from the group consisting of SEQ ID NOs: 72-84.

In another embodiment of the method according to the disclosure, in order to limit reactivity with DNAs present in the sample, a selective extraction for RNA is performed. In this embodiment, each of said one or more primer pairs can be selected independently not only from sets 1) and 2) but also from a primer pair from a third set (“set 3)”) wherein a first primer has a sequence selected from the group consisting of SEQ ID NOs: 92-105 and a second primer has a sequence selected from the group consisting of SEQ ID NOs: 106-111.

In a preferred embodiment of the method according to the disclosure, the reaction mixture further comprises a probe, i.e., an oligonucleotide capable of hybridizing under the reaction conditions of the amplification process to the amplification product constituted by DNA, in order to allow its detection and/or quantification.

In a preferred embodiment of the method according to the disclosure:

a) at least one of the one or more primer pairs is a primer pair from set 1) and the mixture further comprises a probe having a sequence selected from the group consisting of SEQ ID NOs: 46-49; or

b) at least one of the one or more primer pairs is a primer pair from set 2) and the mixture further comprises a probe having a sequence selected from the group consisting of SEQ ID NOs: 85-91; or

c) at least one of the one or more primer pairs is a primer pair from set 3) and the mixture further comprises a probe having a sequence selected from the group consisting of SEQ ID NOs: 112-116.

The three sets of primer pairs described above with the corresponding three probe groups make it possible to detect three alternative regions of the sequence of the mRNA of the UL21.5 gene of hCMV. As it is evident to the person skilled in the art, each primer pair within a set is compatible with the probes that do not have sequence overlap with the primers on the same DNA filament or have overlap with the primers on the complementary filament for no more than 6 nucleotides.

In order to design a probe with ideal characteristics for PCR, such as sensitivity and specificity, one or more nucleotides of the probe can be replaced with corresponding nucleotide analogues. It is possible to use various nucleotide analogues to obtain the desired base pairing properties. These include, among others, peptide nucleic acids (PNA), morpholino, glycol nucleic acids (GNA), threose nucleic acids (TNA), xeno nucleic acids (XNA), and locked nucleic acids (LNA). Moreover, there are modifications aimed at stabilizing the probes by means of molecules that interact with the double strand of DNA.

The probes can also include modifications adapted to allow their detection directly (for example fluorophores, quenchers, metal complexes, etc.) or indirectly (for example biotin, digoxigenin, linker sequences, etc.).

In a preferred but nonlimiting embodiment of the present disclosure, the probes include two modifications, respectively a fluorophore in position 5′ and a quencher in position 3′. In a particularly preferred but nonlimiting embodiment of the disclosure, the fluorophore is 6-FAM and the quencher is TAMRA.

Preferably, the reaction mixture further comprises a primer pair and optionally a probe which are specific for a control sequence other than the hCMV mRNA target sequence. This control sequence is preferably added to the reaction mixture in the form of exogenous nucleic acid, for example a plasmid or a synthetic DNA or the genome of a virus, of a bacterium or of a bacteriophage, in order to constitute an “internal check” to evaluate the integrity of the nucleic acids and the absence of amplification inhibitors in the tested sample.

In a preferred embodiment of the method according to the disclosure, the amplification process is polymerase chain reaction (PCR).

Even more preferably, the amplification process is a quantitative PCR of the realtime type.

In a preferred embodiment of the method according to the disclosure, the antiviral drug capable of interfering with the cleavage and encapsidation of hCMV DNA is a viral terminase complex inhibitor.

In a more preferred embodiment of the method according to the disclosure, the viral terminase complex inhibitor is letermovir or a pharmaceutically acceptable salt thereof.

In another aspect, the present disclosure relates to an oligonucleotide, specific for mRNA of the UL21.5 gene of hCMV, consisting of a sequence selected from the group consisting of SEQ ID NOs: 1-116.

In a preferred embodiment, the oligonucleotide according to the disclosure is a primer having a sequence selected from the group consisting of SEQ ID NOs: 1-45, SEQ ID NOs: 50-84, and SEQ ID NOs: 92-111.

In a preferred embodiment, the oligonucleotide according to the disclosure is a probe having a sequence selected from the group consisting of SEQ ID NOs: 46-49, SEQ ID NOs: 85-91, and SEQ ID NOs: 112-116.

The present disclosure also relates to a kit for the detection and/or quantification of hCMV virion mRNA comprising:

a) at least one primer pair selected from:
a primer pair from a first set (“set 1)”) wherein a first primer has a sequence selected from the group consisting of SEQ ID NOs: 1-12 and a second primer has a sequence selected from the group consisting of SEQ ID NOs: 13-45; or
a primer pair from a second set (“set 2)”) wherein a first primer has a sequence selected from the group consisting of SEQ ID NOs: 50-71 and a second primer has a sequence selected from the group consisting of SEQ ID NOs: 72-84; or
a primer pair from a third set (“set 3)”) wherein a first primer has a sequence selected from the group consisting of SEQ ID NOs: 92-105 and a second primer has a sequence selected from the group consisting of SEQ ID NOs: 106-111; and

optionally

b) an RNA-dependent DNA polymerase;

c) a DNA polymerase.

In a preferred embodiment, the kit according to the disclosure further comprises a probe. For example, in this embodiment of the kit:

a) the at least one primer pair is a primer pair from set 1) and the probe has a sequence selected from the group consisting of SEQ ID NOs: 46-49; or

b) the at least one primer pair is a primer pair from set 2) and the probe has a sequence selected from the group consisting of SEQ ID NOs: 85-91; or

c) the at least one primer pair is a primer pair from set 3) and the probe has a sequence selected from the group consisting of SEQ ID NOs: 112-116.

In a preferred embodiment, the kit according to the disclosure further comprises a primer pair and optionally a probe which are specific for a control sequence other than the hCMV mRNA target sequence.

In a preferred embodiment of the kit according to the disclosure, the control sequence other than the hCMV mRNA target sequence is that of the MS2 bacteriophage with RNA genome, such as for example the one listed in the EBI ENA sequence database with the identification code (ID) V00642 (version 1 of 21 Oct. 1996).

For example, the kit according to the disclosure can comprise a primer pair specific for the genomic RNA of the MS2 phage, having respectively the sequences of SEQ ID NO: 118 and SEQ ID NO: 119 and a probe having the sequence of SEQ ID NO: 120.

The disclosure will be described further with reference to the following nonlimiting example.

### Example: Nonselective Extraction of Nucleic Acids (DNA+RNA) from 1 mL of Plasma and Selective Amplification for mRNA of the UL21.5 Gene of hCMV

This example describes a method for detecting and/or quantifying the mRNA of the UL21.5 gene of hCMV in a non-cellular sample as provided by the disclosure. The test was performed to verify the efficiency of reverse transcription and amplification, the specificity for mRNA of the UL21.5 gene of hCMV and the absence of interference by the corresponding sequences of hCMV genomic DNA.

The method consists in the extraction of the nucleic acids of the sample, reverse transcription/amplification/detection. All the steps of the method were performed automatically by using a model ELITe InGenius® integrated instrument (ELITechGroup S.p.A., code INT030).

Sample Preparation

Artificial samples simulating non-cellular clinical samples were prepared by serial dilution of an hCMV culture of the Merlin strain (ATCC® VR-1590TM), in a matrix constituted by plasma collected in EDTA from healthy donors and tested negative for hCMV DNA with a commercial molecular diagnostic assay (CMV ELITe MGB® Kit, ELITechGroup S.p.A., code RTK015PLD).

The hCMV culture had a concentration of 5×106 PFU (Plaque Forming Unit) of hCMV/mL. The same material was quantified with a commercial molecular diagnostic assay (CMV ELITe MGB® Kit, ELITechGroup S.p.A., code RTK015PLD) and was found to have a concentration, calculated on genomic DNA, of approximately 3×108 International Units (IU) of hCMV/mL.

This concentration in IU higher than that in PFU was expected, since PFU measurement only detects viral particles capable of infecting cells, whereas measurement of the genomic DNA copies also detects the viral particles that are not capable of infecting but contain the viral DNA, and the free viral DNA that is present in the culture supernatant, as occurs in plasma samples of patients with active hCMV infection being treated with antiviral drugs that inhibit the terminase complex of hCMV, such as letermovir.

The simulated samples were prepared by means of four serial dilutions of the hCMV culture having a concentration of 5×106 PFU/mL (Plaque Forming Unit) of hCMV in plasma: 1:100, 1:1,000, 1:10,000, 1:100,000. Each simulated sample was analyzed twice.

Extraction of the Nucleic Acids

For each analyzed simulated sample, 1 mL of sample was transferred into a test tube (Sonication Tube, supplied in the ELITe InGenius® SP 200 Consumable Set product, ELITechGroup S.p.A., code INT032CS) and loaded into the sample area of the ELITe InGenius® instrument.

At the beginning of the extraction step, 10 μL of the CPE-Internal Control product (ELITechGroup S.p.A., code CTRCPE), a stabilized solution that provides exogenous internal control of extraction and inhibition, were dispensed automatically into the extraction cartridge (supplied in the ELITe InGenius® SP 1000 product, ELITechGroup S.p.A., code INT033SP1000) of each sample. The internal control was then processed together with the sample for the entire procedure of extraction and reverse transcription and realtime PCR to demonstrate that the sample had been processed appropriately and that the result of the assay was valid. The target for the internal control is constituted by the genomic RNA of the MS2 bacteriophage, which is not correlated and does not have sequence homologies with hCMV. The formulation of the internal control is ready for use and only needs to be loaded by the operator into the reagent area of the ELITe InGenius® instrument.

The nucleic acids of the samples (DNA and RNA) were extracted automatically by the ELITe InGenius® instrument by using the ELITe InGenius® SP1000 Extraction Kit product (ELITechGroup S.p.A., code INT033SP1000), which uses chaotropic agents, proteases, magnetic balls, solutions of alcohol and water for molecular biology to lyse the sample, capture the nucleic acids, purify them and elute them. The nucleic acids were eluted in 100 μL of water for molecular biology.

### Setup of the Reaction for Reverse Transcription and Realtime Amplification

In the step of reaction setup, the ELITe InGenius® instrument, for each sample, transferred 20 μL of “CMV RNA PCR Mix” mixture (see Table 1) into the well of the PCR cassette (supplied in the ELITe InGenius® PCR Cassette product, ELITechGroup S.p.A., code INT035PCR) placed in the thermal block.

In this example, the CMV RNA PCR Mix contains a primer pair specific for mRNA of the UL21.5 gene of hCMV selected from “set 2)” (SEQ ID NO: 52 and SEQ ID NO: 75) and the compatible probe having SEQ ID NO: 87 modified with 6-FAM in position 5′ and TAMRA in position 3′, since they are designed astride the splice between the exons of the mRNA of the UL21.5 gene of hCMV and are therefore selective for mRNA in an extracted sample in which both DNA and RNA are present. The CMV RNA PCR Mix also contains primers specific for the genomic RNA of the MS2 phage (SEQ ID NO 118 and SEQ ID NO 119) and the corresponding probe (SEQ ID NO 120) modified with 6-FAM in position 5′ and TAMRA in position 3′.

In addition to the primers and probes cited above, the CMV RNA PCR Mix also comprises triphosphate nucleosides, magnesium chloride and the following reagents:

Taq Buffer with KCl (Thermo Fisher Scientific, included in the product Ref EP0404) is a component that establishes the optimum ionic force (potassium chloride) and pH (TRIS-HCl) and provides the substances necessary for the activity of the enzymes (detergents).

RevertAid H Minus Reverse Transcriptase (Thermo Fisher Scientific, Ref EP0451) is a recombinant version of the enzyme of the Moloney Murine Leukemia Virus (M-MuLV). This enzyme has an RNA-dependent and DNA-dependent DNA polymerase activity without RNasi H activity. Thermal stability is increased and can be used for synthesis of the first filament of cDNA at temperatures from 42° C. up to 50° C., achieving a higher specificity.

Platinum® Taq DNA polymerase (Thermo Fisher Scientific, Ref EP0404) is a recombinant version of the DNA polymerase enzyme of the Thermus acquaticus bacterium complexed with an antibody which blocks 5′→3′ DNA polymerase activity at ambient temperature and without 3′→5′ exonucleasic activity to obtain a higher processivity (number of nucleotides added to each bond). DNA polymerase activity was restored with incubation at 95° C. (hot start), obtaining higher specificity and sensitivity.

The formulation of the CMV RNA PCR Mix mixture is presented in

TABLE 1
Formulation of the CMV RNA PCR Mix

| Component | Final concentration in the PCR reaction |
|---|---|
| CMV UL21.5 Forward Primer (SEQ ID NO 52) | 0.5 μM |
| CMV UL21.5 Reverse Primer (SEQ ID NO 75) | 1.3 μM |
| CMV UL21.5 probe (SEQ ID NO 87) | 0.2 μM |
| IC MS2 Forward Primer (SEQ ID NO 118) | 0.05 μM |
| IC MS2 Reverse Primer (SEQ ID NO 119) | 0.13 μM |
| IC MS2 Probe (SEQ ID NO 120) | 0.2 μM |
| dATP | 0.2 mM |
| dCTP | 0.2 mM |
| dGTP | 0.2 mM |
| dTTP | 0.2 mM |
| Magnesium chloride | 4 mM |
| Taq buffer with KCl | 1X |
| RevertAid H Minus Reverse Transcriptase | 3 Units/reaction |
| Platinum ® Taq DNA polymerase | 3 Units/reaction |

The ELITe InGenius® instrument delivers 10 μL of the nucleic acids purified from the sample in the well of the PCR cassette, mixes them with the CMV RNA PCR Mix so as to obtain the final 30 μL of the complete reaction and seals the well with the adapted clear stopper.

### Reaction for Reverse Transcription and Realtime Amplification

The thermal block of the ELITe InGenius® instrument performs the reverse transcription and amplification cycle and the optical system of the instrument detects in real time the fluorescence in the well of the PCR cassette through the clear stopper.

The thermal cycle conditions of the assay for reverse transcription and amplification are listed in Table 2

TABLE 2
Conditions of the RT-PCR thermal cycle

| Description | Cycles | Temperature | Duration |
|---|---|---|---|
| Reverse transcription | 1 | 50° C. | 1200 sec |
| DNApolymerase | 1 | 95° C. | 300 sec |
| activation | | | |
| Amplification | 45 | 95° C. | 10 sec |
| (fluorescence reading) | | 60° C. | 35 sec |

### Interpretation of the Results

At the end of the assay, the software of the ELITe InGenius® instrument automatically runs the interpretation of the results, following preset rules, and creates the reports.

The test required calculation of the standard calibration curve of the assay for the quantification of the samples with realtime PCR. Four serial dilutions of a plasmid DNA with known titer containing, cloned inside it, a sequence that corresponds to part of the cDNA of the UL21.5 gene of hCMV (SEQ ID NO:121) were used for standard curve calculation.

### Results

The test yielded the results summarized in Table 3.

TABLE 3

| | | Complete reaction mix | Incomplete reaction mix |
|---|---|---|---|
| | | (with reverse | (without reverse |
| Sample | Parameter | transcriptase) | transcriptase) |
| Linearity of the series of | R² | 0.9980 | — |
| dilutions | Angular coeff. | −3.2660 | — |
| Target dilution 1:100 | Target copies/mL | 172563 | — |
| | Ct Target | 25.13 | Not determined |
| (5 × 10⁴ PFU of hCMV/ | | | |
| mL) | | | |
| (3 × 10⁶ IU of hCMV/ | | | |
| mL) | | | |
| Target dilution 1:1,000 | Target copies/mL | 17566 | — |
| (5 × 10³ PFU of CMV/ | Ct Target | 28.27 | Not determined |
| mL) | | | |
| (3 × 10⁵ IU of hCMV/ | | | |
| mL) | | | |
| Target dilution 1:10,000 | Target copies/mL | 2130 | — |
| (5 × 10² PFU of CMV/ | Ct Target | 31.17 | Not determined |
| mL) | | | |
| (3 × 10⁴ IU of hCMV/ | | | |
| mL) | | | |
| Target dilution 1:100,000 | Target copies/mL | 143 | — |
| (5 × 10¹ PFU of CMV/ | Ct Target | 35.05 | Not determined |
| mL) | | | |
| (3 × 10³ IU of hCMV/ | | | |
| mL) | | | |

The data in Table 3 demonstrate that the method according to the disclosure makes it possible to detect and quantify virion mRNA of the UL21.5 gene of hCMV with the linear correlation that is typical of molecular assays based on quantification of genomic DNA commonly used to monitor hCMV infection (R²=0.998; angular coefficient=−3.266). The capability of the assay to detect and quantify the presence of mRNA of the UL21.5 gene in virions of hCMV by means of an efficient reaction of reverse transcription and realtime PCR has therefore been verified.

Moreover, absence of interference by the genomic DNA sequences that correspond to the mRNA of the UL21.5 gene that are present in the sample has been verified. At the dilutions examined, hCMV genomic DNA is approximately 15 times more concentrated than UL21.5 mRNA. However, in the controls in which reverse transcriptase was not used and therefore the RNA present in the extracted sample was not amplified, no signal was detected, even for the highest titers (˜3×106 IU of hCMV/mL).

Considering the titer in PFU of hCMV/mL of the reference material in the analysis of the results of this test, quantification of hCMV by means of the mRNA of the UL21.5 gene contained in virions is approximately 3 times higher than the value of the PFU. This result is as expected, since measurement of PFU detects only the viral particles capable of infecting the cells and not the particles that are present but not capable of infecting. Moreover, the virion of hCMV may carry multiple copies of the mRNA of the UL21.5 gene.

This example shows that the method according to the disclosure has provided a quantification of the hCMV contained in the simulated samples that is very similar to the concentration in PFU of hCMV/mL. The concentration in PFU of hCMV/mL is the measurement of viremia when performed with the culture method to identify the infecting viral particles that are present in the sample of the patient and is the most specific indicator of active infection with hCMV.

Taking into account the titer in copies of hCMV genomic DNA/mL of the reference material in the analysis of the results of this test, quantification of hCMV by means of mRNA of UL21.5 contained in virions is 15 times lower than that of copies of hCMV genomic DNA. This result is as expected, since measurement of the copies of genomic DNA also detects the viral genomic DNA that is present in the culture supernatant, as occurs in samples of plasma of patients with active hCMV infection being treated with antiviral drugs that inhibit the terminase complex of hCMV, such as letermovir.

Advantageously, the method according to the disclosure provides a quantification of hCMV that is not influenced by the presence of viral genomic DNA in the culture supernatant, DNA which is not contained in viral particles capable of infecting cells. In the dilutions examined, the concentration of IUs of hCMV is in fact approximately 60 times higher than that of PFUs of hCMV.

In practice it has been found that the method according to the disclosure, as well as the oligonucleotides and the kits useful in said method, fully achieve the intended aim, since they make it possible to perform a quantitative detection of hCMV in non-cellular samples by reverse transcription and realtime PCR of viral mRNAs contained in virions. Therefore, this method can be applied successfully for example in the case of immunodepressed or immunosuppressed patients monitored for the viral load of hCMV and treated with antivirals, including those that cause accumulation of viral DNA. The efficiency of the method is comparable to the one obtained with DNA-based methods and can meet the requirements of current guidelines for monitoring the viral load of hCMV in transplant patients (Kotton et al., Transplantation 2018; 102(6): 900-931, and Emery V et al., Br. J. Haematol. 2013; 162(1): 25-39). Moreover, monitoring of viral mRNA contained in viral particles of hCMV in the sample without the cellular fraction advantageously allows monitoring of the presence of circulating virions of hCMV.

The disclosures in Italian Patent Application No. 102020000007357 from which this application claims priority are incorporated herein by reference.

1-14. (canceled)

15. A method for detection and/or quantification of a human cytomegalovirus (hCMV) virion mRNA in a sample obtained from a patient treated with an antiviral drug capable of interfering with cleavage and encapsidation of hCMV DNA, comprising the steps of:
i) optionally removing from the sample obtained from the patient any cellular component that might be present, obtaining a cell-free sample;
ii) extracting RNA from a cell-free sample obtained from the patient or from the cell-free sample obtained in step i);
iii) forming a reaction mixture by contacting said RNA extracted in step ii) with a solution comprising:
a) one or more primer pairs specific for said hCMV virion mRNA;
b) an RNA-dependent DNA polymerase;
c) a DNA polymerase;
iv) subjecting said reaction mixture to a reverse transcription process under conditions suitable to generate a cDNA corresponding to said hCMV virion mRNA;
v) subjecting said cDNA to an amplification process under conditions adapted to generate an amplification product constituted by DNA;
vi) detecting a presence and/or quantifying the quantity of said amplification product constituted by DNA, as an indication of the presence and/or of the quantity of hCMV virion mRNA in the sample obtained from the patient.

16. The method according to claim 15, wherein each of said one or more primer pairs is independently selected from:
a primer pair from a first set (“set 1)”) wherein a first primer has a sequence selected from a group consisting of SEQ ID NOs: 1-12 and a second primer has a sequence selected from a group consisting of SEQ ID NOs: 13-45; or
a primer pair from a second set (“set 2)”) wherein a first primer has a sequence selected from a group consisting of SEQ ID NOs: 50-71 and a second primer has a sequence selected from a group consisting of SEQ ID NOs: 72-84; or
a primer pair from a third set (“set 3)”) wherein a first primer has a sequence selected from a group consisting of SEQ ID NOs: 92-105 and a second primer has a sequence selected from a group consisting of SEQ ID NOs: 106-111.

17. The method according to claim 16, wherein:
a) at least one of the one or more primer pairs is a primer pair from set 1) and the mixture further comprises a probe having a sequence selected from a group consisting of SEQ ID NOs: 46-49; or
b) at least one of the one or more primer pairs is a primer pair from set 2) and the mixture further comprises a probe having a sequence selected from a group consisting of SEQ ID NOs: 85-91; or
c) at least one of the one or more primer pairs is a primer pair from set 3) and the mixture further comprises a probe having a sequence selected from a group consisting of SEQ ID NOs: 112-116.

18. The method according to claim 15, wherein said amplification process is polymerase chain reaction (PCR).

19. The method according to claim 15, wherein said antiviral drug capable of interfering with the cleavage and encapsidation of hCMV DNA is a viral terminase complex inhibitor.

20. The method according to claim 19, wherein said viral terminase complex inhibitor is letermovir or a pharmaceutically acceptable salt thereof.

21. The method according to claim 15, wherein said sample obtained from the patient is selected from a group consisting of plasma optionally containing an anticoagulant, serum, urine, saliva, oral swab supernatant, cerebrospinal fluid (CSF), bronchoalveolar lavages (BAL), nasopharyngeal lavages, nasopharyngeal aspirates, pharyngeal swab supernatant, tear fluid, vitreous humor, eye swab supernatant, fecal supernatant.

22. The method according to claim 15, wherein said hCMV virion mRNA is an mRNA of a gene or of a coding region (ORF or CDS) of hCMV selected from the group consisting of UL21.5 (SEQ ID NO: 117), UL65 (SEQ ID NO: 122), UL83 (SEQ ID NO: 123), UL106 (SEQ ID NO: 124), UL107 (SEQ ID NO: 125), UL108 (SEQ ID NO: 126), UL109 (SEQ ID NO: 127), UL110 (SEQ ID NO: 128), UL122 (SEQ ID NO: 129), UL123 (SEQ ID NO: 130), TRL/IRL2 (SEQ ID NO: 131), TRL/IRL3 (SEQ ID NO: 132), TRL/IRL4 (SEQ ID NO: 133), TRL/IRL5 (SEQ ID NO: 134), TRL/IRL7 (SEQ ID NO: 135), and TRL/IRL13 (SEQ ID NO: 136).

23. The method according to claim 15, wherein said hCMV virion mRNA is mRNA of the UL21.5 gene (SEQ ID NO: 117).

24. An oligonucleotide consisting of a sequence selected from a group consisting of SEQ ID NOs: 1-116.

25. The oligonucleotide according to claim 24, wherein said oligonucleotide is a primer having a sequence selected from a group consisting of SEQ ID NOs: 1-45, SEQ ID NOs: 50-84, and SEQ ID NOs: 92-111.

26. The oligonucleotide according to claim 24, wherein said oligonucleotide is a probe having a sequence selected from a group consisting of SEQ ID NOs: 46-49, SEQ ID NOs: 85-91, and SEQ ID NOs: 112-116.

27. A kit for detection and/or quantification of hCMV virion mRNA, comprising:
a) at least one primer pair selected from:
a primer pair from a first set (“set 1)”) wherein a first primer has a sequence selected from a group consisting of SEQ ID NOs: 1-12 and a second primer has a sequence selected from a group consisting of SEQ ID NOs: 13-45; or
a primer pair from a second set (“set 2)”) wherein a first primer has a sequence selected from a group consisting of SEQ ID NOs: 50-71 and a second primer has a sequence selected from a group consisting of SEQ ID NOs: 72-84; or
a primer pair from a third set (“set 3)”) wherein a first primer has a sequence selected from a group consisting of SEQ ID NOs: 92-105 and a second primer has a sequence selected from a group consisting of SEQ ID NOs: 106-111; and
optionally
b) an RNA-dependent DNA polymerase; and
c) a DNA polymerase.

28. The kit according to claim 27, wherein:
a) the at least one primer pair is a primer pair from set 1) and the kit further comprises a probe having a sequence selected from a group consisting of SEQ ID NOs: 46-49; or
b) the at least one primer pair is a primer pair from set 2) and the kit further comprises a probe having a sequence selected from a group consisting of SEQ ID NOs: 85-91; or
c) the at least one primer pair is a primer pair from set 3) and the kit further comprises a probe having a sequence selected from a group consisting of SEQ ID NOs: 112-116.


SEQUENCE LISTING (136 sequences)

SEQ ID NO: 1 - DNA, 30 bp, Artificial Sequence
  primer fw rtS2R UL21.5
atcataactcagaaatcgaagcgcagcgtg

SEQ ID NO: 2 - DNA, 30 bp, Artificial Sequence
  fw primer rtS2R UL21.5
atcataatcagaaatcgaagcgcagcgtga

SEQ ID NO: 3 - DNA, 30 bp, Artificial Sequence
  fw primerrt S2R UL21.5
aaatcataatcagaaatcgaagcgcagcgt

SEQ ID NO: 4 - DNA, 30 bp, Artificial Sequence
  fw primer rtS2R UL21.5
aatcataatcagaaatcgaagcgcagcgtg

SEQ ID NO: 5 - DNA, 30 bp, Artificial Sequence
  fw primer rtS2R UL21.5
tcataacttctcagaaatcgaagcgcagcg

SEQ ID NO: 6 - DNA, 30 bp, Artificial Sequence
  fw primer rtS2R UL21.5
atcataacttctcagaaatcgaagcgcagc

SEQ ID NO: 7 - DNA, 30 bp, Artificial Sequence
  fw primer rtS2R UL21.5
tcataacttctcagaaatcgaagcgcatcg

SEQ ID NO: 8 - DNA, 30 bp, Artificial Sequence
  fw primer rtS2R UL21.5
gcggcaccttctcagaaatcgaagcgcagc

SEQ ID NO: 9 - DNA, 30 bp, Artificial Sequence
  fw primer rtS2R UL21.5
cggcaccttctcagaaatcgaagcgcagcg

SEQ ID NO: 10 - DNA, 30 bp, Artificial Sequence
  fw primer rtS2R UL21.5
ggcaccttctcagaaatcgaagcgcagcgt

SEQ ID NO: 11 - DNA, 30 bp, Artificial Sequence
  fw primer rtS2R UL21.5
gcaccttctcagaaatcgaagcgcagcgtg

SEQ ID NO: 12 - DNA, 30 bp, Artificial Sequence
  fw primer rtS2R UL21.5
caccttctcagaaatcgaagcgcagcgtga

SEQ ID NO: 13 - DNA, 30 bp, Artificial Sequence
  rv primer CMV UL21.5 R pr1
ataagggtggtattactaccatcagcgctg

SEQ ID NO: 14 - DNA, 30 bp, Artificial Sequence
  rv primer CMV UL21.5 R pr2
cataagggtggtattactaccatcagcgct

SEQ ID NO: 15 - DNA, 30 bp, Artificial Sequence
  rv pirmer CMV UL21.5 R pr3
cataacctgactgagagttacgttcttgct

SEQ ID NO: 16 - DNA, 30 bp, Artificial Sequence
  rv primer CMV UL21.5 R pr3b
cataacctgactgagagttacgtccttgtt

SEQ ID NO: 17 - DNA, 30 bp, Artificial Sequence
  rv primer CMV UL21.5 R pr4
cataactgactgagagttacgttcttgctg

SEQ ID NO: 18 - DNA, 30 bp, Artificial Sequence
  rv primer CMV UL21.5 R pr5
ataacctgactgagagttacgttcttgctg

SEQ ID NO: 19 - DNA, 30 bp, Artificial Sequence
  rv primer CMV UL21.5 R pr6a
ataaccctgactgagagttacgttcttgct

SEQ ID NO: 20 - DNA, 30 bp, Artificial Sequence
  rv primer CMV UL21.5 R pr6b
ataaccctgactgagagttacgtccttgtt

SEQ ID NO: 21 - DNA, 30 bp, Artificial Sequence
  rv primer CMV UL21.5 R pr7
cataagatccctcctgactgagagttacgt

SEQ ID NO: 22 - DNA, 30 bp, Artificial Sequence
  rv primer CMV UL21.5 R pr8
cataagaccctccctgactgagagttacgt

SEQ ID NO: 23 - DNA, 30 bp, Artificial Sequence
  rv primer CMV UL21.5 R pr12
tcataaccctgactgagagttacgttcttg

SEQ ID NO: 24 - DNA, 30 bp, Artificial Sequence
  rv primer CMV UL21.5 R pr13
ataactccctgactgagagttacgttcttg

SEQ ID NO: 25 - DNA, 30 bp, Artificial Sequence
  rv primer CMV UL21.5 R pr17
ttgctgggggtggtattactaccatcagcg

SEQ ID NO: 26 - DNA, 30 bp, Artificial Sequence
  rv primer CMV UL21.5 R pr18
tcttgctgggggtggtattactaccatcag

SEQ ID NO: 27 - DNA, 30 bp, Artificial Sequence
  rv primer CMV UL21.5 R pr19
gttcttgctgggggtggtattactaccatc

SEQ ID NO: 28 - DNA, 30 bp, Artificial Sequence
  rv primer CMV UL21.5 R pr20
ttacgttcttgctgggggtggtattactac

SEQ ID NO: 29 - DNA, 30 bp, Artificial Sequence
  rv primer CMV UL21.5 R pr21
gagttacgttcttgctgggggtggtattac

SEQ ID NO: 30 - DNA, 30 bp, Artificial Sequence
  rv primer CMV UL21.5 R pr22
tgagagttacgttcttgctgggggtggtat

SEQ ID NO: 31 - DNA, 30 bp, Artificial Sequence
  rv primer CMV UL21.5 R pr23
actgagagttacgttcttgctgggggtggt

SEQ ID NO: 32 - DNA, 28 bp, Artificial Sequence
  rv primer CMV UL21.5 R pr25
ccctgactgagagttacgttcttgctgg

SEQ ID NO: 33 - DNA, 30 bp, Artificial Sequence
  rv primer CMV UL21.5 R pr36
agtaatcttcgtctccgtcggtggtggacc

SEQ ID NO: 34 - DNA, 30 bp, Artificial Sequence
  rv primer CMV UL21.5 R pr37
ccccggagtaatcttcgtctccgtcggtgg

SEQ ID NO: 35 - DNA, 30 bp, Artificial Sequence
  rv primer CMV UL21.5 R pr38
atactccccggagtaatcttcgtctccgtc

SEQ ID NO: 36 - DNA, 30 bp, Artificial Sequence
  rv primer CMV UL21.5 R pr39
cgtcatactccccggagtaatcttcgtctc

SEQ ID NO: 37 - DNA, 30 bp, Artificial Sequence
  rv primer CMV UL21.5 R pr40
caaaacgtcatactccccggagtaatcttc

SEQ ID NO: 38 - DNA, 30 bp, Artificial Sequence
  rv primer CMV UL21.5 R pr41
aatcaaaacgtcatactccccggagtaatc

SEQ ID NO: 39 - DNA, 30 bp, Artificial Sequence
  rv primer CMV UL21.5 R pr43
tctccgtctgtaatcaaaacgtcatactcc

SEQ ID NO: 40 - DNA, 30 bp, Artificial Sequence
  rv primer CMV UL21.5 R pr44
gctgccatctccgtctgtaatcaaaacgtc

SEQ ID NO: 41 - DNA, 30 bp, Artificial Sequence
  rv primer CMV UL21.5 R pr45
ttcgctgccatctccgtctgtaatcaaaac

SEQ ID NO: 42 - DNA, 30 bp, Artificial Sequence
  rv primer CMV UL21.5 R pr46
tgatgttcgctgccatctccgtctgtaatc

SEQ ID NO: 43 - DNA, 30 bp, Artificial Sequence
  rv primer CMV UL21.5 R pr47
gttgctgatgttcgctgccatctccgtctg

SEQ ID NO: 44 - DNA, 30 bp, Artificial Sequence
  rv primer CMV UL21.5 R pr48
tttgtggttgctgatgttcgctgccatctc

SEQ ID NO: 45 - DNA, 30 bp, Artificial Sequence
  rv primer CMV UL21.5 R pr49
cagtcttttgtggttgctgatgttcgctgc

SEQ ID NO: 46 - DNA, 25 bp, Artificial Sequence
  probe CMV UL21.5 S pr1
tgacggtggaacaacccagtaccag

SEQ ID NO: 47 - DNA, 24 bp, Artificial Sequence
  probe CMV UL21.5 S pr2
acggtggaacaacccagtaccagc

SEQ ID NO: 48 - DNA, 25 bp, Artificial Sequence
  probe CMV UL21.5 S pr3
acggtggaacaacccagtaccagcg

SEQ ID NO: 49 - DNA, 25 bp, Artificial Sequence
  probe CMV UL21.5 S pr4
cggaacaacccagtaccaccagcgc

SEQ ID NO: 50 - DNA, 30 bp, Artificial Sequence
  primer fw CMV UL21.5 F pr7
aaatcataacatggctcggaggctatggat

SEQ ID NO: 51 - DNA, 30 bp, Artificial Sequence
  primer fw CMV UL21.5 F pr8
aaatcataacagcatggctcggaggctatg

SEQ ID NO: 52 - DNA, 30 bp, Artificial Sequence
  primer fw CMV UL21.5 F pr9
taaatcataatcagcatggctcggaggcta

SEQ ID NO: 53 - DNA, 30 bp, Artificial Sequence
  primer fw CMV UL21.5 F pr19
cagcagcagcgacgccgacaagacctcagc

SEQ ID NO: 54 - DNA, 30 bp, Artificial Sequence
  primer fw CMV UL21.5 F pr20
cagcagcgacgccgacaagacctcagcatg

SEQ ID NO: 55 - DNA, 30 bp, Artificial Sequence
  primer fw CMV UL21.5 F pr21
cagcgacgccgacaagacctcagcatggct

SEQ ID NO: 56 - DNA, 30 bp, Artificial Sequence
  primer fw CMV UL21.5 F pr22
gcgacgccgacaagacctcagcatggctcg

SEQ ID NO: 57 - DNA, 30 bp, Artificial Sequence
  primer fw CMV UL21.5 F pr23
acgccgacaagacctcagcatggctcggag

SEQ ID NO: 58 - DNA, 30 bp, Artificial Sequence
  primer fw CMV UL21.5 F pr24
ccgacaagacctcagcatggctcggaggct

SEQ ID NO: 59 - DNA, 30 bp, Artificial Sequence
  primer fw CMV UL21.5 F pr25
acaagacctcagcatggctcggaggctatg

SEQ ID NO: 60 - DNA, 30 bp, Artificial Sequence
  primer fw CMV UL21.5 F pr26
gacctcagcatggctcggaggctatggatc

SEQ ID NO: 61 - DNA, 30 bp, Artificial Sequence
  primer fw CMV UL21.5 F pr27
ctcagcatggctcggaggctatggatcttg

SEQ ID NO: 62 - DNA, 30 bp, Artificial Sequence
  primer fw CMV UL21.5 F pr28
cagcatggctcggaggctatggatcttgag

SEQ ID NO: 63 - DNA, 30 bp, Artificial Sequence
  fw primer CMV UL21.5 F pr29
catggctcggaggctatggatcttgagctt

SEQ ID NO: 64 - DNA, 30 bp, Artificial Sequence
  fw primer CMV UL21.5 F pr30
tggctcggaggctatggatcttgagcttac

SEQ ID NO: 65 - DNA, 30 bp, Artificial Sequence
  fw primer CMV UL21.5 F pr31
tcggaggctatggatcttgagcttactagc

SEQ ID NO: 66 - DNA, 30 bp, Artificial Sequence
  fw primer CMV UL21.5 F pr32
gaggctatggatcttgagcttactagccgt

SEQ ID NO: 67 - DNA, 30 bp, Artificial Sequence
  fw primer CMV UL21.5 F pr33
gctatggatcttgagcttactagccgtgac

SEQ ID NO: 68 - DNA, 30 bp, Artificial Sequence
  fw primer CMV UL21.5 F pr34
tggatcttgagcttactagccgtgaccttg

SEQ ID NO: 69 - DNA, 30 bp, Artificial Sequence
  fw primer CMV UL21.5 F pr35
gatcttgagcttactagccgtgaccttgac

SEQ ID NO: 70 - DNA, 30 bp, Artificial Sequence
  fw primer CMV UL21.5 F pr36
ttgagcttactagccgtgaccttgacggtg

SEQ ID NO: 71 - DNA, 30 bp, Artificial Sequence
  fw primer CMV UL21.5 F pr37
agcttactagccgtgaccttgacggtggct

SEQ ID NO: 72 - DNA, 30 bp, Artificial Sequence
  rv primer CMV UL21.5 R pr9
aataaatcataaccaccgtcacgctgcgct

SEQ ID NO: 73 - DNA, 30 bp, Artificial Sequence
  rv primer CMV UL21.5 R pr10
aataaatcataatccaccgtcacgctgcgc

SEQ ID NO: 74 - DNA, 30 bp, Artificial Sequence
  rv primer CMV UL21.5 R pr11
aataaatcataattccaccgtcacgctgcg

SEQ ID NO: 75 - DNA, 30 bp, Artificial Sequence
  rv primer CMV UL21.5 R pr14
aataaatcataaccaccgtcacgctgagct

SEQ ID NO: 76 - DNA, 30 bp, Artificial Sequence
  rv primer CMV UL21.5 R pr15
ataaatcataaccaccgtcacgctgcgctt

SEQ ID NO: 77 - DNA, 30 bp, Artificial Sequence
  rv primer CMV UL21.5 R pr50
cgctggtactgggttgttccaccgtcacgc

SEQ ID NO: 78 - DNA, 30 bp, Artificial Sequence
  rv primer CMV UL21.5 R pr51
gctggtactgggttgttccaccgtcacgct

SEQ ID NO: 79 - DNA, 30 bp, Artificial Sequence
  rv primer CMV UL21.5 R pr52
ctggtactgggttgttccaccgtcacgctg

SEQ ID NO: 80 - DNA, 30 bp, Artificial Sequence
  rv primer CMV UL21.5 R pr53
tggtactgggttgttccaccgtcacgctgc

SEQ ID NO: 81 - DNA, 30 bp, Artificial Sequence
  rv primer CMV UL21.5 R pr54
ggtactgggttgttccaccgtcacgctgcg

SEQ ID NO: 82 - DNA, 30 bp, Artificial Sequence
  rv primer CMV UL21.5 R pr55
gtactgggttgttccaccgtcacgctgcgc

SEQ ID NO: 83 - DNA, 30 bp, Artificial Sequence
  rv primer CMV UL21.5 R pr56
tactgggttgttccaccgtcacgctgcgct

SEQ ID NO: 84 - DNA, 30 bp, Artificial Sequence
  rv primer CMV UL21.5 R pr57
actgggttgttccaccgtcacgctgcgctt

SEQ ID NO: 85 - DNA, 23 bp, Artificial Sequence
  probe CMV UL21.5 S pr5
cttgacggtggctttggcggcac

SEQ ID NO: 86 - DNA, 24 bp, Artificial Sequence
  probe CMV UL21.5 S pr6
cggtggctttggcggcaccttctc

SEQ ID NO: 87 - DNA, 24 bp, Artificial Sequence
  probe CMV UL21.5 S pr7
tggctttggcggcaccttctcaga

SEQ ID NO: 88 - DNA, 25 bp, Artificial Sequence
  probe CMV UL21.5 S pr8
ctttggcggcaccttctcagaaatc

SEQ ID NO: 89 - DNA, 25 bp, Artificial Sequence
  probe CMV UL21.5 S pr9
tttggcggcaccttctcagaaatcg

SEQ ID NO: 90 - DNA, 25 bp, Artificial Sequence
  probe CMV UL21.5 S pr10
tggcggcaccttctcagaaatcgaa

SEQ ID NO: 91 - DNA, 25 bp, Artificial Sequence
  probe CMV UL21.5 S pr11
cggcaccttctcagaaatcgaagcg

SEQ ID NO: 92 - DNA, 30 bp, Artificial Sequence
  fw primer CMV UL21.5 F pr38
aacacaaagaaaatcaagccaaagaaaatg

SEQ ID NO: 93 - DNA, 30 bp, Artificial Sequence
  fw primer CMV UL21.5 F pr39
aaagaaaatcaagccaaagaaaatgaaaag

SEQ ID NO: 94 - DNA, 30 bp, Artificial Sequence
  fw primer CMV UL21.5 F pr40
gaaaatcaagccaaagaaaatgaaaagaag

SEQ ID NO: 95 - DNA, 30 bp, Artificial Sequence
  fw primer CMV UL21.5 F pr41
ttcaagccaaagaaaatgaaaagaagattc

SEQ ID NO: 96 - DNA, 30 bp, Artificial Sequence
  fw primer CMV UL21.5 F pr42
ccaaagaaaatgaaaagaagattcagtaac

SEQ ID NO: 97 - DNA, 30 bp, Artificial Sequence
  fw primer CMV UL21.5 F pr43
gaaaatgaaaagaagattcagtaacagcag

SEQ ID NO: 98 - DNA, 30 bp, Artificial Sequence
  fw primer CMV UL21.5 F pr44
aaatgaaaagaagattcagtaacagcagac

SEQ ID NO: 99 - DNA, 30 bp, Artificial Sequence
  fw primer CMV UL21.5 F pr45
aagaagattcagtaacagcagaccccaagg

SEQ ID NO: 100 - DNA, 30 bp, Artificial Sequence
  fw primer CMV UL21.5 F pr46
ttcagtaacagcagaccccaagggttaacg

SEQ ID NO: 101 - DNA, 30 bp, Artificial Sequence
  fw primer CMV UL21.5 F pr47
aacagcagaccccaagggttaacgattatg

SEQ ID NO: 102 - DNA, 30 bp, Artificial Sequence
  fw primer CMV UL21.5 F pr48
agcagaccccaagggttaacgattatgttg

SEQ ID NO: 103 - DNA, 30 bp, Artificial Sequence
  fw primer CMV UL21.5 F pr49
accccaagggttaacgattatgttgactac

SEQ ID NO: 104 - DNA, 30 bp, Artificial Sequence
  fw primer CMV UL21.5 F pr50
aagggttaacgattatgttgactaccttgt

SEQ ID NO: 105 - DNA, 30 bp, Artificial Sequence
  fw primer CMV UL21.5 F pr58
aaagctgtaaggttttgctctaaaaacacc

SEQ ID NO: 106 - DNA, 30 bp, Artificial Sequence
  rv primer CMV UL21.5 R pr70
tagttagacaggctatgggaagaaaccgtg

SEQ ID NO: 107 - DNA, 30 bp, Artificial Sequence
  rv primer CMV UL21.5 R pr71
aggctagttagacaggctatgggaagaaac

SEQ ID NO: 108 - DNA, 30 bp, Artificial Sequence
  rv primer CMV UL21.5 R pr72
gggaaggctagttagacaggctatgggaag

SEQ ID NO: 109 - DNA, 30 bp, Artificial Sequence
  rv primer CMV UL21.5 R pr73
ctcacgggaaggctagttagacaggctatg

SEQ ID NO: 110 - DNA, 30 bp, Artificial Sequence
  rv primer CMV UL21.5 R pr74
aaactctcacgggaaggctagttagacagg

SEQ ID NO: 111 - DNA, 30 bp, Artificial Sequence
  rv primer CMV UL21.5 R pr75
cataaactctcacgggaaggctagttagac

SEQ ID NO: 112 - DNA, 26 bp, Artificial Sequence
  probe CMV UL21.5 S pr12
cttttgtattcggcacgcgaaacacg

SEQ ID NO: 113 - DNA, 25 bp, Artificial Sequence
  probe CMV UL21.5 S pr13
tgtattcggcacgcgaaacacggtt

SEQ ID NO: 114 - DNA, 25 bp, Artificial Sequence
  probe CMV UL21.5 S pr14
tattcggcacgcgaaacacggtttc

SEQ ID NO: 115 - DNA, 25 bp, Artificial Sequence
  probe CMV UL21.5 S pr15
ttcggcacgcgaaacacggtttctt

SEQ ID NO: 116 - DNA, 25 bp, Artificial Sequence
  probe CMV UL21.5 S pr16
cacgcgaaacacggtttcttcccat

SEQ ID NO: 117 - RNA, 512 nt, Human cytomegalovirus
ucagcagcgacgccgacaagaccucagcauggcucggaggcuauggaucuugagcuuacuagccgugaccuugacgguggcuuuggcggcaccuucucagaaaucgaagcgcagcgugacgguggaacaacccaguaccagcgcugaugguaguaauaccacccccagcaagaacguaacucucagucaggggggguccaccaccgacggagacgaagauuacuccggggaguaugacguuuugauuacagacggagauggcagcgaacaucagcaaccacaaaagacugaugaacacaaagaaaaucaagccaaagaaaaugaaaagaagauucaguaacagcagaccccaaggguuaacgauuauguugacuaccuuguuuuuuauuaaaaagcuguaagguuuugcucuaaaaacaccccgccuccggucuuuuuucuuuuguauucggcacgcgaaacacgguuucuucccauagccugucuaacuagccuucccgugagaguuuaug

SEQ ID NO: 118 - DNA, 28 bp, Artificial Sequence
  fw primer IC MS2 F
cgtacgtcaggtcggtactaacatcaag

SEQ ID NO: 119 - DNA, 28 bp, Artificial Sequence
  rv primer IC MS2 R
gcgatatgttgcacgttgtctggaagtt

SEQ ID NO: 120 - DNA, 25 bp, Artificial Sequence
  probe IC MS2 S
ccgtctgtcgtatccagctgcaaac

SEQ ID NO: 121 - DNA, 212 bp, Artificial Sequence
  cDNA cDNA UL21.5
gaattcgacgccgacaagacctcagcatggctcggaggctatggatcttgagcttactagccgtgaccttgacggtggctttggcggcaccttctcagaaatcgaagcgcagcgtgacggtggaacaacccagtaccagcgctgatggtagtaataccacccccagcaagaacgtaactctcagtcagggggggtccaccaccgacgaattc

SEQ ID NO: 122 - RNA, 309 nt, Human cytomegalovirus
ccaucaucaucucaggcucuaucgguaccaucguugucaucugaaaaaaaaacugccucacccaccugcguaaaaacaccaucuuuccggaggugcgguaagacgggcaaauacggucgugccgaggcaaaaaaaacgcaccaucgacaccacacccucaugagcaccaccugucgguguuggucguccuccaucguucucuacgaacaucucgacgcccgggugacggacgacggcaagacgucccggagaagacgguguucucucgggcgguacgcucucuggaucuauaauaucuauaguagcuaa

SEQ ID NO: 123 - RNA, 1686 nt, Human cytomegalovirus
auggagucgcgcggucgccguugucccgaaaugauauccguacugggucccauuucggggcacgugcugaaagccguguuuagucgcggcgauacgccggugcugccgcacgagacgcgacuccugcagacggguauccacguacgcgugagccagcccucgcugaucuugguaucgcaguacacgcccgacucgacgccaugccaccgcggcgacaaucagcugcaggugcagcacacguacuuuacgggcagcgagguggagaacgugucggucaacgugcacaaccccacgggccgaagcaucugccccagccaggagcccaugucgaucuauguguacgcgcugccgcucaagaugcugaacauccccagcaucaacgugcaccacuacccgucggcggccgagcgcaaacaccgacaccugcccguagcugacgcugugauucacgcgucgggcaagcagauguggcaggcgcgucucacggucucgggacuggccuggacgcgucagcagaaccaguggaaagagcccgacgucuacuacacgucagcguucguguuucccaccaaggacguggcacugcggcacguggugugcgcgcacgagcugguuugcuccauggagaacacgcgcgcaaccaagaugcaggugauaggugaccaguacgucaagguguaccuggaguccuucugcgaggacgugcccuccggcaagcucuuuaugcacgucacgcugggcucugacguggaagaggaccugacgaugacccgcaacccgcaacccuucaugcgcccccacgagcgcaacggcuuuacgguguugugucccaaaaauaugauaaucaaaccgggcaagaucucgcacaucaugcuggauguggcuuuuaccucacacgagcauuuugggcugcugugucccaagagcaucccgggccugagcaucucagguaaccuguugaugaacgggcagcagaucuuccuggagguacaagccauacgcgagaccguggaacugcgucaguacgaucccguggcugcgcucuucuuuuucgauaucgacuugcugcugcagcgcgggccucaguacagcgagcaccccaccuucaccagccaguaucgcauccagggcaagcuugaguaccgacacaccugggaccggcacgacgagggugccgcccagggcgacgacgacgucuggaccagcggaucggacuccgacgaagaacucguaaccaccgagcgcaagacgccccgcgucaccggcggcggcgccauggcgggcgccuccacuuccgcgggccgcaaacgcaaaucagcauccucggcgacggcgugcacgucgggcguuaugacacgcggccgccuuaaggccgaguccaccgucgcgcccgaagaggacaccgacgaggauuccgacaacgaaauccacaauccggccguguucaccuggccgcccuggcaggccggcauccuggcccgcaaccuggugcccaugguggcuacgguucagggucagaaucugaaguaccaggaauucuucugggacgccaacgacaucuaccgcaucuucgccgaauuggaaggcguauggcagcccgcugcgcaacccaaacgucgccgccaccggcaagacgccuugcccgggccaugcaucgccucgacgcccaaaaagcaccgagguuga

SEQ ID NO: 124 - RNA, 378 nt, Human cytomegalovirus
augaugaccgaccgcacggagcgacgccgucgacugacccacgcggcauguacgccguccgcgaacaaccaaaggacgacccgucuccccccgcacccggguuuuuucucuuggucgaacccggcuugcgacgacggguuguuccuuuaccggacgacggucagccgcgggguugauacccagcgacggcgucgcucccacccggguuucuucucuugcagguaccacccgucgacugucagccucgcgaggagacaccgcggaccggggaaacggauaaguuuacgaacagaaaucucaaaagacgcugacccgauaaguaccgucacggagacacggugguuuuuauugaauuuccaguguaucgagccaccguga

SEQ ID NO: 125 - RNA, 453 nt, Human cytomegalovirus
aagcuuccuuacagcauaacuguaacauacgaucaucgaacaucuccucacauaccuuucuccucucacauaggaaaacaaaacucuuuuuuuucuuuccugucaaggaaaaaaucaauguaccaccacaucacuuucugcucggucccggcgacggaugggcgcgcacgcggacaaagacccaccggccacuuccacuuauuuuuuguuguuaaucgucuucucccccgcacgcggagcaccaccaagccuagcugcucauucgcucaaccagucacaccgcgcacgcgagaaggggccgggguccgcgggcaccgccgccggaggcgcgguucccucucucuaauucccuggaaaacaaguaaugacaaacaaaaagacgacaaaaggucucuauucugcuacaugagagaaauuauagcuguuggcaauuuuucaaaauacauguuauaa

SEQ ID NO: 126 - RNA, 372 nt, Human cytomegalovirus
ggucaucgucguccacgacgacgugucuauagcgugcggugugaucauugugucgagccagagaaagcgcgccucgcacgacguuugcggaucggcucgcggguguguggaauuccuaagaacauaaucagcuggucgucuuucuuugauguguuguugucgucgaggucuugcuucguuuucuuuuuucuuuuuagucgauggaacuuuucuucgguacggguucuuguuauggaagcuuguguuuucgaacaugaauucgaaaaaauaaaaaggccuaucuucguuucaaaaaaaggacagauaucaaucuucuuaacuuauaucaugguaaauucagaauccuauggugucuuauuaucucuaaaguag

SEQ ID NO: 127 - RNA, 297 nt, Human cytomegalovirus
augauacacgacuaccacuggaggagaggaaaacggagaaaacucaucgugaugauggaaacaagaaugaugauaauaaugaugacgauaaagccaccacacgauauauacagagaacaacaaaaccaccacgacgaugaaacaaaacgcucaaccaaacauuguguaaccgcuacgcauccguggccgcggccggcggccucuuguuuucguuguccccuucgcgggggacaucaucgccgccgacccgcgugugcccugccucacggcugguccgugaugaacucuugcucguaa

SEQ ID NO: 128 - RNA, 384 nt, Human cytomegalovirus
ccuuugaaaaccaagccuauugauaauaaucuaccccauagaacaggauauaaucaagcauccaaacaacaaacagcgccgccgugccuccuccagaggacggugacgacguaugucugucgucucccuucucgcucuggcuucuggggcucuccuccucuccccaucaucuucuccuucgucccagacggacggcuaucgguucgcgcgcucgucuuuucuucgaugugcgcagguuucgucaucggagaagaagaccgcgaccguaucauaaugaugauaauaauaauacauucaccgaccaucuuuauccucuuuuguuguuccaaagagaaaagaaaaaaaaaacaagcagccacucuaaccaucacacucacgaguuaa

SEQ ID NO: 129 - RNA, 1236 nt, Human cytomegalovirus
augcugccccucaucaaacaggaagacaucaagcccgagcccgacuuuaccauccaguaccgcaacaagauuaucgauaccgccggcuguaucgugaucucugauagcgaggaagaacagggugaagaagucgaaacccgcggugcuaccgcgucuuccccuuccaccggcagcggcacgccgcgagugaccucucccacgcacccgcucucccagaugaaccacccuccucuucccgaucccuugggccggcccgaugaagauaguuccucuucgucuuccuccuccugcaguucggcuucggacucggagagugaguccgaggagaugaaaugcagcaguggcggaggagcauccgugaccucgagccaccaugggcgcggcgguuuugguggcgcggccuccuccucucugcugagcugcggccaucagagcagcggcggggcgagcaccggaccccgcaagaagaagagcaaacgcaucuccgaguuggacaacgagaaggugcgcaauaucaugaaagauaagaacacccccuucugcacacccaacgugcagacucggcggggucgcgucaagauugacgaggugagccgcauguuccgcaacaccaaucgcucucuugaguacaagaaccugcccuucacgauucccaguaugcaccagguguuagaugaggccaucaaagccugcaaaaccaugcaggugaacaacaagggcauccagauuaucuacacccgcaaucaugaggugaagagugagguggaugcggugcggugucgccugggcaccaugugcaaccuggcccucuccacucccuuccucauggagcacaccaugcccgugacacauccacccgaaguggcgcagcgcacagccgaugcuuguaacgaaggcgucaaggccgcguggagccucaaagaauugcacacccaccaauuaugcccccguuccuccgauuaccgcaacaugaucauccacgcugccacccccguggaccuguugggcgcucucaaccugugccugccccugaugcaaaaguuucccaaacaggucauggugcgcaucuucuccaccaaccaggguggguucaugcugccuaucuacgagacggccgcgaaggccuacgccguggggcaguuugagcagcccaccgagaccccucccgaagaccuggacacccugagccuggccaucgaggcagccauccaggaccugaggaacaagucucaguaa

SEQ ID NO: 130 - RNA, 1597 nt, Human cytomegalovirus
ucagaucgccuggagacgccauccacgcuguuuugaccuccauagaagacaccgggaccgauccagccuccgcggccgggaacggugcauuggaacgcggauuccccgugccaagagugacauggaguccucugccaagagaaagauggacccugauaauccugacgagggcccuuccuccaaggugccacggcccgagacacccgugaccaaggccacgacguuccugcagacuauguugaggaaggagguuaacagucagcugagucugggagacccgcuguuuccagaguuggccgaagaaucccucaaaacuuuugaacaagugaccgaggauugcaacgagaaccccgagaaagauguccuggcagaacucgucaaacagauuaagguucgaguggacauggugcggcauagaaucaaggagcacaugcugaaaaaauauacccagacggaagagaaauucacuggcgccuuuaauaugaugggaggauguuugcagaaugccuuagauaucuuagauaagguucaugagccuuucgaggagaugaaguguauugggcuaacuaugcagagcauguaugagaacuacauuguaccugaggauaagcgggagauguggauggcuuguauuaaggagcugcaugaugugagcaagggcgccgcuaacaaguuggggggugcacugcaggcuaaggcccgugcuaaaaaggaugaacuuaggagaaagaugauguauaugugcuacaggaauauagaguucuuuaccaagaacucagccuucccuaagaccaccaauggcugcagucaggccauggcggcacugcagaacuugccucagugcuccccugaugagauuauggcuuaugcccagaaaauauuuaagauuuuggaugaggagagagacaaggugcucacgcacauugaucacauauuuauggauauccucacuacauguguggaaacaauguguaaugaguacaaggucacuagugacgcuuguaugaugaccauguacgggggcaucucucucuuaagugaguucugucgggugcugugcugcuaugucuuagaggagacuagugugaugcuggccaagcggccucugauaaccaagccugagguuaucaguguaaugaagcgccgcauugaggagaucugcaugaaggucuuugcccaguacauucugggggccgauccucugagagucugcucuccuaguguggaugaccuacgggccaucgccgaggagucagaugaggaagaggcuauuguagccuacacuuuggccaccgcuggugucagcuccucugauucucuggugucacccccagaguccccuguacccgcgacuaucccucuguccucaguaauuguggcugagaacagugaucaggaagaaagugagcagagugaugaggaagaggaggagggugcucaggaggagcgggaggacacugugucugucaagucugagccagugucugagauagaggaaguugccccagaggaagaggaggauggugcugaggaacccaccgccucuggaggcaagagcacccacccuauggugacuagaagcaaggcugaccaguaa

SEQ ID NO: 131 - RNA, 348 nt, Human cytomegalovirus
acgucccugguagacgggguagggggaucuaccagcccagggaucgcgucuuucgccgccacgcugcuucaccgauauccaauaaacccauccccucgccacgacgucuccgcguaucuuuguagccucaagaauccguccccacguccacccaucccgagcacuccacacgccauaacaaaccacggacacgacaaaugcaugcaaacuucucauuuauugugucuacuacucuguguugcuacagggagugaagagggugaaggcaaagaaaaaaaaaaggaacaaaauaauagauuagcagaaggaauaauccgugcgaccgagcuugugcuucuuuucuuauaa

SEQ ID NO: 132 - RNA, 345 nt, Human cytomegalovirus
auguauuguuuucuuuuuuuacagaaagauacuuuuuuucaugagcaauuccucgcccggcgccggcaugccgagguggggccacugcgaucagcggcaugccgacgccgacccggggaucuuggauucaccguuuucucucuucucucucuacauacagaccggguggcaggagcgguaaggaaucaucgucgucuuucauucuucgaugauuaugguaauacuaaaucuuaucuaggagcauauacaucuaagauuggaguacuaguagucguuugugguuucuauuuuuuuuuauauuuaucuaugacaguuuuucuguuuuucguuuugauaauaauauaa

SEQ ID NO: 133 - RNA, 513 nt, Human cytomegalovirus
augcagcaugcacgcguguauguaugcaucgccucuccuccgucccgacuaccaucagcaguaccacugccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauag

SEQ ID NO: 134 - RNA, 345 nt, Human cytomegalovirus
augcauacauacacgcgugcaugcugcauggguggaugguacggccgggagacgcggaagagaaacucacauaaaaaggugacaaaaagagcgguugaaaaaagaaaacaagauucgaccagacagaagagaaggaccggggcuuggcgacccuuccacgacugcuguugucaucucggcuccuccgucuucucccggccacgggcggcuaagucaccgccguucuccccauccguccgagcgccgaccgaccagccggccgauucgcccgccggggcuucuggagaacgccggggcagcagcgaucuggggaagcugcuaaaccccugcguuuuuauaugguag

SEQ ID NO: 135 - RNA, 249 nt, Human cytomegalovirus
augaaagcaagaggcagccgagagaaugccaguaagagacgaccaucgcagacacaguacgauacucaucuuagaacgaaccagcgaauaaccaucacacguacagcagaaucucaugaacuagucaaccaacgucauaaaaucuucacacaaucguuuuugcgaacuuuuaggaaccagcaagucaacaaaagacuaacaaagaaaaaccaucuuggaauuaaaaaaaguagcaucguuaccuuauga

SEQ ID NO: 136 - RNA, 444 nt, Human cytomegalovirus
auggacuggcgauuuacgguuauguggacgauacuaauauccgcguuaucagaaagcugcaaucaaaccuguuccugucaaugucccuguaguacuaccguuaacuauuccacuaguacugagacagccacaucaacauacaguacaacaguuaucagcaauaaaagcacuucagaaucuauaaauugcucuacugcaacugcaccagcaaccaccguuucuacaaaaccgucgaaaacaaccacacagauauccacaacgacaaauacaaacguugagacuaccacauguaccaacaccaccacgaccguuacuugugaugguuucaauuauacaguccauaaaagaugcgaccgcaguuacgagguaaucaacguaacaggauacguugguggcaacauaacucuaaaaaaugcaaucagacugagaaauggcacaauguag